(19) United States
(12) Patent Application Publication
(10) Pub. No.: US 2025/0049383 A1
(43) Pub. Date: Feb. 13, 2025

(54) CONTACTLESS WOUND MONITORING

(71) Applicant: KONINKLIJKE PHILIPS N.V., EINDHOVEN (NL)

(72) Inventors: Tobias WISSEL, Lübeck (DE); Sven KRÖNKE-HILLE, Hamburg (DE)

(21) Appl. No.: 18/718,467

(22) PCT Filed: Dec. 9, 2022
(86) PCT No.: PCT/EP2022/085185

§ 371 Date: Jun. 11, 2024

Related U.S. Application Data
(60) Provisional application No. 63/291,559, filed on Dec. 20, 2021.

(51) Int. Cl. A61B 5/00; A61B 5/01

(52) U.S. Cl. CPC A61B 5/445; A61B 5/0035; A61B 5/0077; A61B 5/015; A61B 5/7282; A61B 2562/0233; A61B 2576/02

(57) ABSTRACT

A device includes a light source, a camera, a memory and a processor. The light source emits emission light to a wound at a plurality of wavelengths. The camera detects backscattered light generated based on the emission light. The memory stores instructions. The processor executes the instructions. When executed by the processor, the instructions cause the device to record an image based on the backscattered light; quantify a degree of infection of the wound based on the image, and output an indication of the degree of infection.

### BACKGROUND

Wounds which are not healing may indicate severe underlying disease patterns. Impediments for wound healing include wound size, ischemia, and infection. Infected wounds are typically examined for staging and treatment selection via summarizing indices such as the wound, ischemia, foot infection (wifi) classification system index. Bacterial colonization deprives the chronic wound of oxygen and nutrients and can prolong an inflammatory phase, i.e., prevent the wound from healing even when perfusion deficits have been treated. Therefore, frequent perfusion/oxygenation monitoring needs to be accompanied by measures for monitoring of the infection status over time to pinpoint root causes of the chronic wound, to select care options and to respond to changes with relevant treatment. A positive prognosis for a patient requires a targeted treatment along with monitoring of disease development and treatment impact over time.

Current methods for quantifying infection status require biopsies, organism culturing, bulky hardware or suffer from confounding factors outside an in-vitro context. Current methods are generally inapplicable to a monitoring use case involving frequent measurements when a result is typically not available for several days after biopsies and organism culturing. Alternative, optical approaches are either invasive and require the injection of infrared (IR) dyes or fluorescent agents, or leverage vibrational spectroscopic imaging of substrates colonized by sampled bacteria. Vibrational imaging attempts to determine the exact type and number of bacteria by comparing the spectral signature of the bacteria to a large reference database of pathogens for molecular identification. While in-vitro spectroscopy still takes several hours, corresponding in-vivo attempts usually suffer from a weak signal and overlapping confounding factors. Furthermore, vibrational spectroscopic imaging systems are typically coupled to fiber optical probes or confocal microscopes which makes the imaging approach very local, bulky and therefore impractical for bedside monitoring of larger wound surfaces.

As a result of the background described above, there is an open need for rapid and contactless/remote monitoring of infection status.

### SUMMARY

According to an aspect of the present disclosure, a device includes a light source, a camera, a memory and a processor. The light source emits emission light to a wound at a plurality of wavelengths. The camera detects backscattered light generated based on the emission light. The memory stores instructions. The processor executes the instructions. When executed by the processor, the instructions cause the device to record an image based on the backscattered light; quantify a degree of infection of the wound based on the image, and output an indication of the degree of infection.

According to another aspect of the present disclosure, a method for quantifying a degree of an infection includes emitting, by a light source of a device, emission light to a wound at a plurality of wavelengths; detecting, by a camera of the device, backscattered light generated based on the emission light; recording an image based on the backscattered light; quantifying a degree of infection of the wound based on the image, and outputting, from the device, an indication of whether the wound is infected.

According to another aspect of the present disclosure, a system includes a light source, a camera, a memory and a processor. The light source emits emission light to a wound at a plurality of wavelengths. The camera detects backscattered light generated based on the emission light. The memory stores instructions. The processor executes the instructions. When executed by the processor, the instructions cause the system to record an image based on the backscattered light; quantify a degree of infection of the wound based on the image, and output an indication of the degree of infection.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1A illustrates a system for contactless wound monitoring, in accordance with a representative embodiment.

FIG. 1B illustrates a device for contactless wound monitoring, in accordance with a representative embodiment.

FIG. 1C illustrates a controller for contactless wound monitoring, in accordance with a representative embodiment.

FIG. 1D illustrates another device for contactless wound monitoring, in accordance with a representative embodiment.

FIG. 1E illustrates another device for contactless wound monitoring, in accordance with a representative embodiment.

FIG. 2 illustrates a method for contactless wound monitoring, in accordance with a representative embodiment.

FIG. 3 illustrates another method for contactless wound monitoring, in accordance with a representative embodiment.

FIG. 4 illustrates a computer system, on which a method for contactless wound monitoring is implemented, in accordance with another representative embodiment.

### DETAILED DESCRIPTION

In the following detailed description, for the purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. The defined terms are in addition to the technical and scientific meanings of the defined terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only and is not intended to be limiting. As used in the specification and appended claims, the singular forms of terms ‘a’, ‘an’ and ‘the’ are intended to include both singular and plural forms, unless the context clearly dictates otherwise. Additionally, the terms “comprises”, and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to”, “coupled to”, or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims. Moreover, descriptions of well-known apparatuses and methods may be omitted so as to not obscure the description of the example embodiments. Such methods and apparatuses are within the scope of the present disclosure.

As described herein, contactless and rapid monitoring of wound infection status may be provided using multispectral optical imaging or hyperspectral optical imaging. Optical backscatter from the multispectral optical imaging or hyperspectral optical imaging is influenced by the presence of bacteria in the wound, such that the optical backscatter across a spectrum of different wavelengths may be exploited to determine status of the wound. The optical backscatter may be recorded via a camera and then analyzed by a controller to determine whether a wound is infected.

As described herein, wounds can be monitored for infections using a single contactless device or system. The device or system is configured for quick use, such that the device or system may be used on an ad-hoc basis or on a persistent basis.

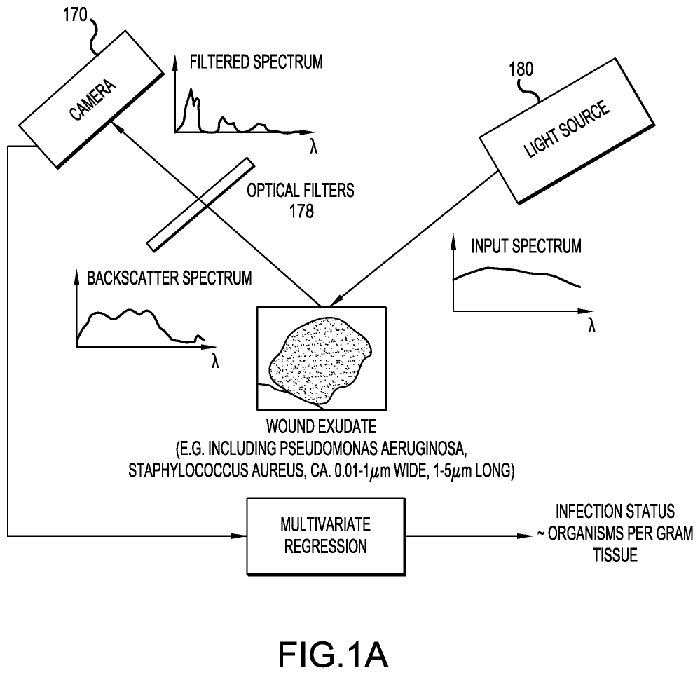

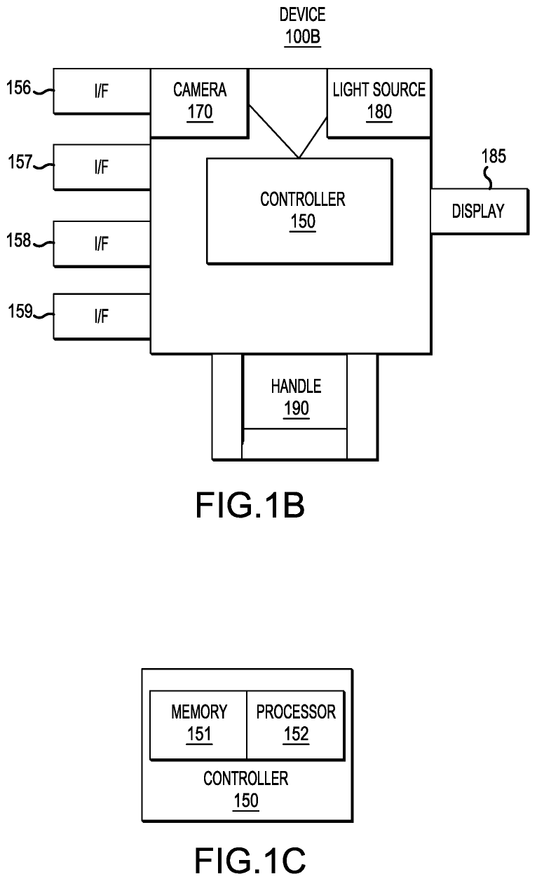

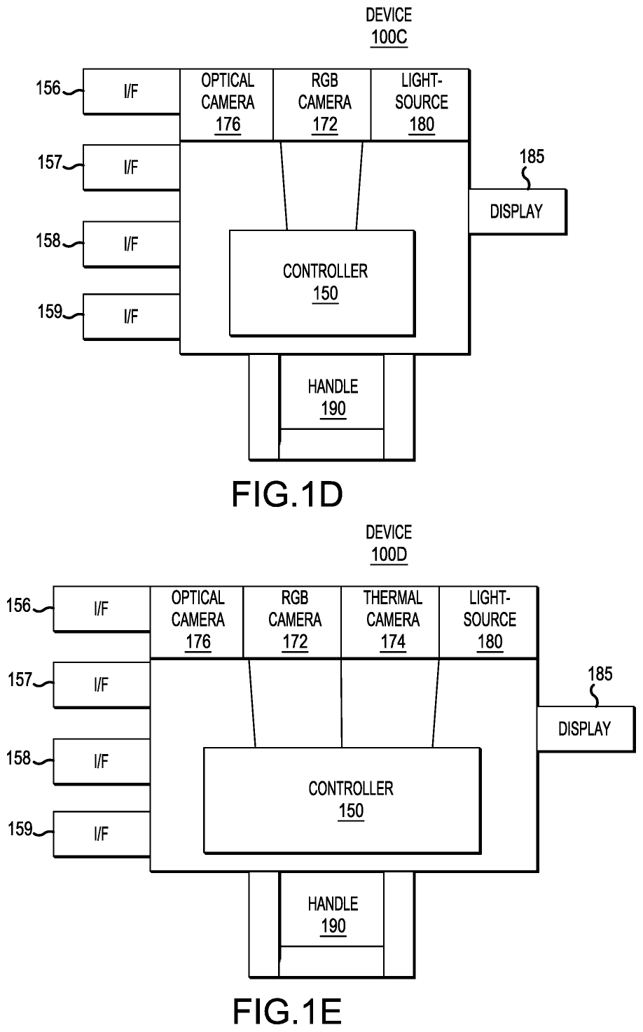

FIG. 1A illustrates a system for contactless wound monitoring, in accordance with a representative embodiment.

The system in FIG. 1A is a system for contactless wound monitoring and includes components that may be provided together as in a device, or that may be distributed as shown in the system of FIG. 1A. The system in FIG. 1A includes a camera 170, optical filters 178 and a light source 180. The camera 170, the optical filters 178 and/or the light source 180 are an imaging unit. The system in FIG. 1A provides monitoring via remote optical sensing of light backscattered from the wound. Although not shown in FIG. 1A, the system in FIG. 1A may also include a memory that stores instructions and a processor that executes the instructions, such as by a controller described herein. The combination of a memory and a processor described herein comprise an inference unit. Additionally, although not shown in FIG. 1A, the system in FIG. 1A may include a display and/or another output mechanism. The display and/or other output mechanism comprise an infection status output unit. The display and/or other output mechanism is or includes an interface used to output at least one of a visual signal or an audible signal. The output visual signal or audible signal serve as an indication of whether the wound is infected, and in some embodiments, the extent of infection.

The camera 170 may be capable of recording a multi-spectral backscattered image. The camera 170 is configured to spatially resolve and spectrally resolve backscatter. The camera 170 is configured to determine, from the recorded backscattered image, which light intensity is recorded at which wavelength. The multi-variate information per pixel in the camera image provides backscattered light intensity information in different spectral ranges/wavelengths. The camera 170 is configured to determine spectral per-pixel resolution, either by sensor design or by time multiplex as sequentially spectral bins are filtered out of the incoming light by fast changing subsequent bandpass filters. The camera 170 may also output one image or a sequence of images.

The system in FIG. 1A irradiates the wound area with light from the light source 180. The light from the light source 180 covers a band of different wavelengths. The optical filters 178 filter the backscattered light reflected from the wound. The camera 170 records a spatially resolved and spectrally resolved diffuse reflectance image based on the backscattered light reflected from the wound. The diffuse reflectance image may also be temporally resolved by the camera 170. The camera 170 may be configured specifically to be sensitive to the selected range of relevant wavelengths of the backscattered light reflected from the wound. For example, wavelengths of emitted light from the light source 180 and/or wavelengths of the backscattered light to be detected by the camera 170 may be selected via a user interface or may be predetermined before use such as when the camera 170 and/or the light source 180 are assembled. Examples of wavelengths and bands appropriate for the camera 170 and the light source 180 include wavelengths in the visible range (VIS range), wavelengths in the near infrared range (NIR range), and wavelengths in the higher NIR/IR range. The near infrared range penetrates a wound well without being absorbed by water. The higher NIR/IR range is useful in probing water absorption insofar as liquid wound exudate may be an indicator of infection.

The light source 180 may be a multi-wavelength light source or a hyper-wavelength light source. A multi-wavelength light source may be configured to provide light that can be detected by a multi-spectral camera. A hyper-wavelength light source may be configured to provide light that can be detected by a hyper-spectral camera. A multi-wavelength light source may emit light at fewer wavelengths (e.g., a smaller band or a smaller set) than a hyper-spectral wavelength light source. In embodiments based on FIG. 1A, the light source 180 emits light to a wound at an input spectrum. The light from the camera results in backscattered reflective light at a backscatter spectrum when reflected from the wound. The backscattered reflective light is passed through the optical filters 178 to produce a filtered spectrum, and the backscattered reflective light in the filtered spectrum is detected by the camera 170. The backscattered reflective light has a backscatter spectrum different from the input spectrum. The backscattered reflective light in the filtered spectrum is a subset of the backscattered reflective light.

The backscattered reflective light in the filtered spectrum is input to a multivariate regression model which outputs an infection status. The multivariate regression model may be implemented via instructions stored in a memory and executed by a processor, such as by a controller described herein. The multivariate regression model may be obtained from empirical evidence. The ground truth for the regression module may be derived from alternative measurement means. An output of the infection status may be, for examples, a binary output such as green/red or infected/not infected or a granular output such as organisms per gram of tissue. All types of infected or non-infected wounds will result in backscattered light. The backscatter light detected by the cameras describe herein is detected based on changes in the spectrum that indicate the presence of scatterers or absorbers which have optical properties of listed bacteria. The backscattered spectrum may also be measured and evaluated relative to a non-wound reference location. An example of the wound exudate which results in the backscattered reflective light detected by cameras described herein is Pseudomonas aeruginosa Staphylococcus aureus, CA with a width of 0.01-1 μm and a length of 1-5 μm.

In FIG. 1A, the recorded spectrum for the backscatter may be normalized based on the amounts of emitted light at each wavelength from the light source 180. The recorded spectrum for the backscatter may alternatively be normalized by recording a camera image (“dark image”) with the light source 180 switched off, as this provides information about light which is entering the camera 170 after passing through the filter and which is from sources other than the light source 180. The multivariate regression model may then be applied to infer infection status from the reflectance image. The reflectance image from the camera 170 is spatially resolved, spectrally resolved, and in some embodiments temporally resolved, so as to yield a multivariate signal per pixel. The status may be output such as by illustrating the status on a display for a user. The normalization and the application of the multivariate regression model may be performed by one or more processors executing instructions, such as by a controller described herein. The output of the status may be via a display and/or another output mechanism which outputs information perceptible to a human.

FIG. 1B illustrates a device for contactless wound monitoring, in accordance with a representative embodiment.

The device 100B includes a controller 150, a first interface 156, a second interface 157, a third interface 158, a fourth interface 159, a camera 170, a light source 180, and a handle 190. The device 100B may be a single, integrated device that can be used to detect wound infection status quickly by emitting light from the light source 180, detecting the backscattered reflective light at the camera 170, and processing the backscattered reflective light at the controller 150.

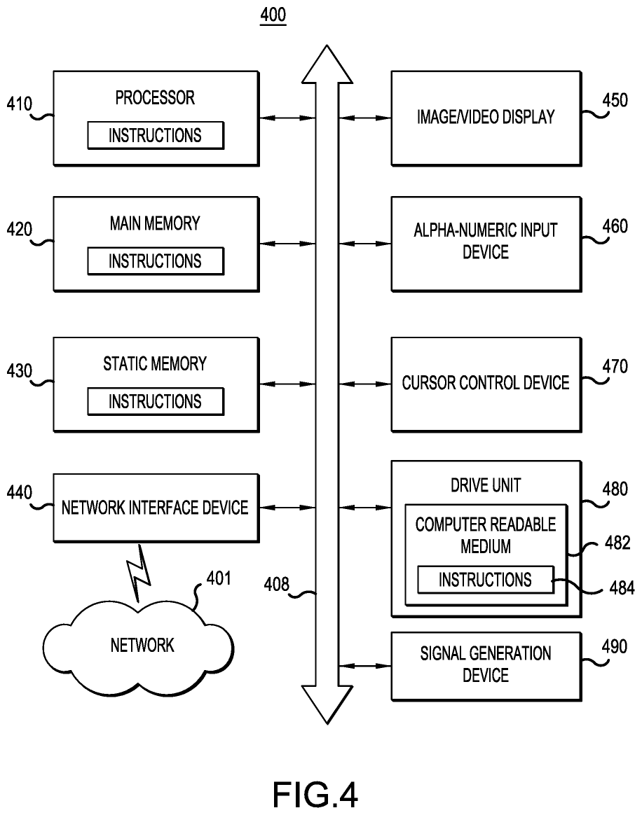

The controller 150 is further depicted in FIG. 1C, and includes at least a memory 151 that stores instructions and a processor 152 that executes the instructions. The controller 150 may operate as an inference unit. A computer that can be used to implement the controller 150 is depicted in FIG. 4, though the device 100B may include more additional elements than depicted in the combination of FIG. 1B, FIG. 1C and FIG. 4.

The first interface 156, the second interface 157, the third interface 158 and the fourth interface 159 may include ports, disk drives, wireless antennas, or other types of receiver circuitry. The first interface 156, the second interface 157, the third interface 158 and the fourth interface 159 may also include user interfaces such as thumbwheels, buttons, knobs and interactive touch-displays.

The camera 170 may be or include multiple cameras, such as an optical camera, a RGB camera, and/or a thermal camera. The camera 170 is a sensor sensitive to the desired band of wavelengths which are either predetermined or selected via a user interface. The output of the camera 170 is a spectrally and spatially resolved image of the wound area.

The light source 180 may alone be considered an imaging unit, or may be considered an imaging unit in combination with the camera 170. The light source 180 irradiates a broad band of wavelengths onto the wound area. The incident spectrum of the light source 180 is either predetermined, or is determined from frequent or even constant measurements. Knowledge of the incident spectrum is used to calibrate camera images recorded by the camera 170 against the input signal. For example, the camera images recorded by the camera 170 may comprise the backscatter spectrum, and may be normalized by knowledge of the amount of light emitted at each wavelength of the incident spectrum.

The light from the light source 180 is diffusely backscattered from the wound and recorded by the camera 170.

Although not shown in FIG. 1B, the device 100B may include one or more optical filters. The optical filters may include polarizing filters and/or wavelength filters. The recording path may include optical filters to avoid a dominance of the specular reflection and to focus on a set of the most relevant wavelengths.

The handle 190 is representative of handles or similar mechanisms by which a user may hold and move a portable electronic device.

Although not illustrated, the device 100B in FIG. 1B may also include a display and/or may be configured to connect to and communicate with a display. A display included in the device 100B may be a display typical of a handheld mobile device such as a mobile phone, and thus may be or include an LCD-type or LED-type display. A display not included in the device 100B but usable by the device 100B may be or include a monitor such as a computer monitor, an augmented reality display, or another screen configured to display electronic imagery. A display usable by the device 100B may also be or include an interactive touch screen configured to display prompts to users and collect touch input from users.

FIG. 1C illustrates a controller for contactless wound monitoring, in accordance with a representative embodiment.

The controller 150 includes a memory 151 and a processor 152. The memory 151 stores instructions which are executed by the processor 152. The processor 152 executes the instructions. When the controller 150 is provided as a stand-alone unit, the controller 150 may also include one or more of the first interface 156, the second interface 157, the third interface 158, and the fourth interface 159, such as to connect the controller 150 to other elements of a device or system.

The controller 150 may perform some of the operations described herein directly and may implement other operations described herein indirectly. For example, the controller 150 may directly process the backscatter light detected from the wound, and determine based on the backscatter light whether the wound is infected. The controller 150 may also directly control emissions of light by the light source 180. The controller 150 may indirectly control other operations such as by generating and transmitting content to be displayed on an external display. Accordingly, the processes implemented by the controller 150 when the processor 152 executes instructions from the memory 151 may include steps not directly performed by the controller 150.

In embodiments herein, the controller 150 may control the camera 170, the light source 180, other types of cameras described below, and the processing of the multivariate regression model. The controller 150 may be used to provide functions additional to the core functions described herein. For example, when a reference spectrum is available, the reference spectrum may be incorporated into the inference process via normalization of the measurement spectrum in the pre-processing stage or the reference spectrum may be passed to the regression model as an extra input in order to identify the calibration function in a manner that is data-driven from empirical experience.

The regression model is trained to operate as a spatially resolved classifier insofar as the chemical and structural composition of wounds (whether infected or not) differs from normal skin tissue. An example of the regression model is a convolutional neural network trained to segment the wound area given the spatially resolved backscatter spectrum as input. The regression model may also be trained to incorporate a reference backscatter spectrum of healthy skin tissue of the same patient. Thereby, the wound area can be calculated by the controller 150 implementing the regression model without requiring an RGB/grey-value camera. The wound area estimate may then be used for predicting a mean pathogen density as a single crisp measure for the infection extent. The mean pathogen density may be expressed as pathogen number per area.

FIG. 1D illustrates another device for contactless wound monitoring, in accordance with a representative embodiment.

In FIG. 1D, the device 100C includes a controller 150, the first interface 156, the second interface 157, the third interface 158, the fourth interface 159, the optical camera 176, the light source 180 and the handle 90. The device 100C also includes a RGB camera 172.

A variety of additional or alternative features may be provided for the embodiments of FIG. 1A, FIG. 1B, and FIG. 1C. For example, in the in the embodiment of FIG. 1D, a device 100C includes the RGB camera 172. The imaging system of the device 100C may include the optical camera 176, the light source 180 and the RGB camera 172.

The RGB camera 172 is representative of an RGB/grey-value camera with an approximately same field-of-view as the backscatter detector provided by the optical camera 176. The images acquired by the RGB camera 172 may be used to automatically delineate the wound via, for example, a convolutional neural network trained to segment wounds. The images may also be used to calculate the area of the wound, and subsequently pass the calculated wound area to the inference unit for predicting the bacteria density. The bacteria of interest are bacteria with sizes in or above an incident wavelength range, as these are the bacteria which influence the backscatter behavior. The backscattered light proportion per wavelength may be a function of the wound tissue properties and the chemical composition, number and size of the organisms.

FIG. 1E illustrates another device for contactless wound monitoring, in accordance with a representative embodiment.

In FIG. 1E, the device 100D includes a controller 150, the first interface 156, the second interface 157, the third interface 158, the fourth interface 159, the optical camera 176, the RGB camera 172, the light source 180 and the handle 90. The device 100D also includes a thermal camera 174.

A variety of additional or alternative features may be provided for the embodiments of FIG. 1A, FIG. 1B, FIG. 1C and FIG. 1D. For example, in the embodiment of FIG. 1E, a device 100D includes the thermal camera 174. The imaging system of the device 100D may include the optical camera 176, the RGB camera 172, the light source 180 and the thermal camera 174.

The thermal camera 174 may operate as a temperature measurement device. For example, the thermal camera may be or include a thermographic sensor configured to capture a thermographic profile of the wound. The thermal camera 174 may also capture the body surface area temperature outside of the wound and/or the room temperature, as a reference. The thermal camera 174 is incorporated into the device 100D as an element to ensure realization of the device 100D as a fully contactless device. Temperature measurements from the thermal camera 174 may add additional information on the inflammatory status and metabolic activity of the wound. A measurement process based on thermography using the thermal camera 174 and optical sensing using the optical camera 176 may include the acquisition of a reference image from a healthy skin patch, such as a skin patch that is relatively devoice of hair and relatively planar. The reference image may serve as a reference to rule out major confounding factors such as current skin surface temperature based on seasonal individual biases, skin type, skin age etc.

In the embodiments based on FIG. 1A, FIG. 1B, FIG. 1C, FIG. 1D and FIG. 1E, output by a display may be binary or may be based on ranges or even individual numerical values. An infection measure may be computed based on a single camera frame. In some embodiments, however, one or more infection measures may be computed based on multiple camera frames. A camera may capture multiple frames at a sampling rate which is predetermined or set dynamically. A display may output the current infection extent as the target value relative to a time-series showing the infection extent from some or all previous measurements, so that the output is a time-series of a plurality of indications of whether the wound is infected over a period of time. A time-series may provide a basis for inferring trends and potentially adapting the treatment strategy. The time-series infection-extent evolution plot may be either provided for the whole wound or for a manually selected region of interest in the wound if a spatially resolved infection extent regressor is used. The infection measure may be measured on various time scales, such as several times a day, over several days, or continuously by letting the camera run and continuously produce an infection measure. Continuous measurements may be performed analogous to a sliding window, such as by using a fixed number of the current and most recent frames as inputs to continuously compute a single infection measure. A time-series of images may be provided on a millisecond (ms) scale, wherein multiple images are recorded milliseconds apart from one another. A time-series of infection measurement outputs may, however, be continually produced in real time, or intermittently several times a day, or over several days.

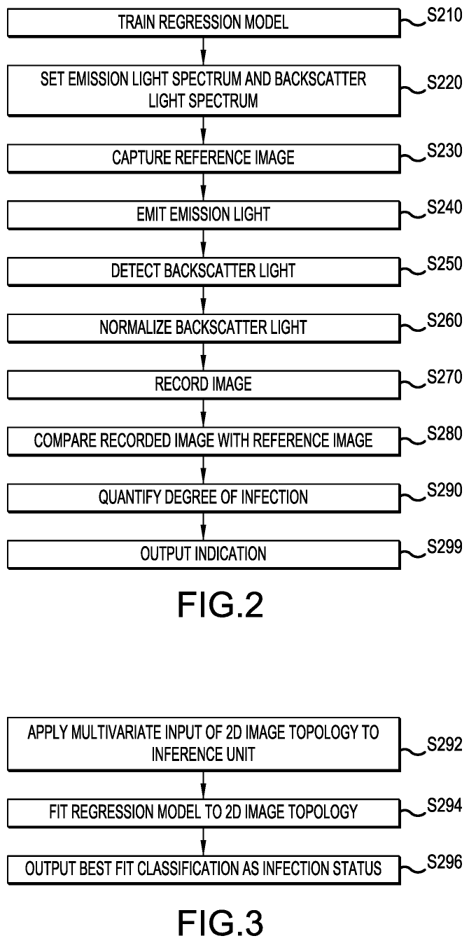

FIG. 2 illustrates a method for contactless wound monitoring, in accordance with a representative embodiment.

The method of FIG. 2 may be performed by the system in FIG. 1A, by the device 100B in FIG. 1B, by the device 100C in FIG. 1D, or by the device 100D in FIG. 1E. The method of FIG. 2 may be performed under the control of the controller 150 in any of these embodiments.

At S210, a regression model is trained. The regression model may be trained for application to images. An example of such a trained regression model is a trained neural network. The regression model may be trained using empirical optical measurements which are linked to target values obtained from clinical standard methods. The regression model may also be trained using Monte-Carlo backscatter simulations from tissue models including different amounts of pathogen scatterers. Alternatively, training of the regression model may be supported by use of such Monte-Carlo backscatter simulations from tissue models including different amounts of pathogen scatterers. The trained regression model may output targets classifications that are binary, such as infection/no infection, or that are ranges, such as a discrete number reflective of ranges of organisms-per-gram-tissue.

At S220, an emission light spectrum and a backscatter light spectrum are each set. An interface on the device 100B, the device 100C or the device 100D may be used to select a range of wavelengths of the backscattered light to use in detecting the backscattered light. The method of FIG. 2 may therefore include accepting a selection of an emission light spectrum to use as emission light and accepting a selection of wavelengths of the backscattered light to use in recording the image.

At S230, a reference image is captured. The reference image may be an image of skin where no wound is present, and is preferably an image of skin that is relatively planar and hairless.

At S240, emission light is emitted. The emission light is emitted at the emission light spectrum set at S220.

At S250, backscatter light is detected. The backscatter light is detected at the backscatter light spectrum set at S220. The backscatter signal will depend on the chemical composition, number and size of organism in the wound. While the impact of the chemical composition and size and/or shape on the backscatter intensity spectrum will mainly reflect the presence of organisms, the number of scatterers in the wound exudate will scale the backscatter response with the grade of infection. These aspects are reflected by the Rayleigh equation, reproduced below as Equation (1). In the Rayleigh equation, the backscattered intensity over a spectrum of different wavelengths λ (at a given distance R to the scatterer and scattering angle θ) depends on the refractive index n as well as the sixth power of the particle diameter d.

$$I = {I}_{0} \frac{1 + {cos}^{2} θ}{2 {R}^{2}} {( \frac{2 π}{λ} )}^{4} {( \frac{{n}^{2} - 1}{{n}^{2} + 2} )}^{2} {( \frac{d}{2} )}^{6} \qquad Equation ( 1 )$$

Since the size of bacterial organism is within or larger than the wavelength band used for monitoring (e.g. including Pseudomonas aeruginosa, Staphylococcus aureus, ca. 0.1-1 μm wide, 1-5 μm long), principles of Mie scattering would however be predominant. However, leaving all other parameters but the particle diameter fixed, the backscatter response (independent of the model that describes it) is higher in the presence (or at a higher number) of scatterers that are larger than the wavelength compared to regimes with scatterers smaller than the incident wavelength.

At S260, backscatter light is normalized. For a specific wavelength or spectral bin of emitted light, the backscattered light may be normalized by the amount of emitted light at the wavelength/bin. Intensities may vary from light source to light source. Part of the normalization may include a recording of an image with the light source switched off, to obtain a reference of the background illumination to be subtracted before relating the leftover backscatter to the source light.

At S270, an image is recorded. Although not specified in FIG. 2, the backscatter light detected at S250 may be filtered to the backscatter light spectrum set at S220 before the image is recorded at S270. That is, the method of FIG. 2 may include filtering the backscattered light before recording the image at S270. The camera 170 described herein also spectrally resolves the recorded image of the backscatter light, and may also temporally resolve the recorded image.

At S280, the recorded image from S270 is compared with the reference image from S230.

After S280, the regression model trained at S210 may be applied to features extracted from the image recorded at S270. For example, a backscatter image may be pre-processed before information is input to the regression model. Pre-processing may involve spatial filtering or temporal filtering. Moreover, an image may comprise a sequence of images, so that temporal features such as pulsation as seen in PPG can be deducted from the images. Local indications for infection may be detected as well as global indications for infection, and application of the regression model to a full image may result in one indication of infection being output. Feature extraction may also involve, for example, a spatial masking of relevant wound area so that the extracted global optical backscatter information can be tailored to the relevant area. Additionally, although not required, a thermographic profile of the wound may be input to the regression model.

Pre-processing may also be provided in the form of a known method to estimate physical or optical properties from the backscattered light. Examples of the physical or optical properties which can be estimated by known pre-processing methods which may be performed consistent with the teachings herein include estimates of hemoglobin or water content, such as from e.g. inverse Monte-Carlo model optimization.

The regression model may be applied by a controller 150, and specifically by a processor executing instructions from a memory. The controller 150 may be or include an inference unit that takes a multivariate input and translates the multivariate input to a target value describing the infection status. The multivariate input includes the spectrally resolved images and may include a temperature measure as well as a reference spectrum from the reference image taken at S230. The controller 150 may exploit the 2-dimensional image topology by convolutional architectures to fit a nonlinear function to empirical data.

The target value defines the type of the inference problem and may be one of the following options, described as follows from basic to elaborate. The most basic target value may be a binary value which classified an entire image into infected or not infected. The next most basic target value may be a classification for the entire image into a measure of the extent of infection. The extent of infection may be proportional or equal to organisms-per-gram-tissue, and may be discretized into coarse ranges such as not-infected, slightly infected, moderately infected, highly infected or highly infected, depending on clinical implications. In more elaborate schemes, the regression model may output a classification for each pixel or group of pixels, either as a binary classification or as a relative measure of the extent of infection. An indication of whether the wound is infected may be output for each of a plurality of subsets of pixel values used to generate an image.

Empirical data and ground truth for the regression model may be obtained from alternative mechanisms which are considered highly reliable, e.g., so-called gold standards. The alternative mechanisms may not be capable of the rapid and remote monitoring made possible by the teachings herein, but yield a clinically established/accepted measure of higher detail than required according to the teachings herein. As examples, the traditional mechanisms used to establish ground truth may not be capable of rapid and remote monitoring due to the time required for analysis, the nature of the bulky/contact-based device(s) used for such mechanisms, and the limited ability to take only local measurements.

As examples of the empirical data used to obtain the regression model, tissue biopsy and culturing may yield densities per type of bacterium per wound. Densities per type may be expressed as organisms-per-gram-tissue. The data of densities per type may be joined into a single organism density to obtain a binary target. As described above, binary targets may be applicable to entire images, but not for individual pixel values or subsets of pixel values in the images. As another example, vibrational spectroscopy may yield one or more number(s) per measurement location, either in-vivo or in-vitro. As with densities per type, the results of vibrational spectroscopy may be joined into a single density. The measurement locations may be (e.g. manually) registered with the camera image recorded as reference image at S230 and then used subsequently for inference. The information measured on a subset of locations across the image/wound area may be “smeared” across the image, such as by using convolutional filters, so as to obtain a ground truth of the full resolution. The smearing technique assumes that the infection status does not vary abruptly across the wound surface, and the technique may be more valid when the ground truth measure is quantized into coarse ranges compared to discrete numerical outputs.

The methodologies used to collect empirical data for the ground truth are each aimed at resolving the pathogen type, whereas the output from the multivariate regression model used herein may be less detailed. To build a robust inference module for the infection status/extent, the multivariate regression model is trained on cases with a variety of wounds such that sufficient variability in the concentration, type, shape, size of pathogens and other confounding factors is covered. The factors in the variability of wounds used to establish the ground truth may be factors not otherwise taken into account in the reference image taken at S230 or any dedicated model, and the factors in the variability of wounds used to establish the ground truth may exclude factors such as skin type, wound age, depth.

At S299, an indication is output. The indication may be output as a display, as an audio tone, by a color indication such as by individual light-emitting diodes (LEDs), or by other mechanisms perceptible to a human. The output is an indication of infection status. The inference result may be finally displayed to the user, such as on the device 100B that outputs the light, captures the backscatter spectrum, and applies the multivariate regression model. Depending on the chosen target value, the display may be realized as a colormap overlay that indicates the regression output/infection status per pixel on top of the wound image. An indication of whether the wound is infected may be output for each of a plurality of subsets of pixel values used to generate an image. The display may alternatively be realized as a color range/bar indicator which visually classifies the infection status into an evaluated context. For example, the display may output green for no infection and red for highly infected and action needed.

In a bedside or home monitoring scenario, the infection status indicator can be combined with a threshold and warning system that triggers the patient to see an expert for more detailed analysis

FIG. 3 illustrates another method for contactless wound monitoring, in accordance with a representative embodiment.

At S292, a multivariate input of 2-dimensional (2D) image topology is input to an inference unit. A camera records backscattered light from the wound. At each pixel, the camera may resolve the sensed backscattered light intensity into contributions stemming from different wavelength. In other words, multiple variables are derived from across pixels (space) and possibly time in the instance of multiple camera images. In space the pixels are subject to a specific 2-dimensional topology. An example of a specific 2-dimensional topology is a 2-dimensional regular grid of pixels, where a pixel neighborhood is defined. The multivariate input may also be or include other types of information not particularly related to the camera. Other types of information may include, for example, temperature, reference spectrum from areas outside of the wound, patient-specific parameters such as age and skin type, and so on.

At S294, a regression model is fit to the 2-dimensional image topology. A regression model is fit to data that empirically describes the functional relationship between multivariate backscatter signal and infection status. The regression model thereby fits a non-linear function to the data. By fitting the nonlinear function, the regression model (predicting an indicator either per pixel or for the whole image) may exploit the neighborhood information provided by the 2-dimensional topology of the image. The regression model may predict an indicator either per pixel or for an entire image. An example of S294 is a 2-dimensional convolutional neural network fusing information from a local neighborhood as defined by its filter kernels.

At S296, best fit classifications are output from the regression model as an infection status.

The method of FIG. 3 may be performed as part of S290 in FIG. 2, such as to quantify a degree of infection as the infection status from the best fit classification at S296.

FIG. 4 illustrates a computer system, on which a method for contactless wound monitoring is implemented, in accordance with another representative embodiment.

FIG. 4 illustrates a computer system, on which a method for contactless wound monitoring is implemented, in accordance with another representative embodiment.

Referring to FIG. 4, the computer system 400 includes a set of software instructions that can be executed to cause the computer system 400 to perform any of the methods or computer-based functions disclosed herein. The computer system 400 may operate as a standalone device or may be connected, for example, using a network 401, to other computer systems or peripheral devices. In embodiments, a computer system 400 performs logical processing based on digital signals received via an analog-to-digital converter.

In a networked deployment, the computer system 400 operates in the capacity of a server or as a client user computer in a server-client user network environment, or as a peer computer system in a peer-to-peer (or distributed) network environment. The computer system 400 can also be implemented as or incorporated into various devices, such as the devices described herein including a stationary device or a mobile device, a mobile computer, a laptop computer, a tablet computer, or any other machine capable of executing a set of software instructions (sequential or otherwise) that specify actions to be taken by that machine. The computer system 400 can be incorporated as or in a device that in turn is in an integrated system that includes additional devices. In an embodiment, the computer system 400 can be implemented using electronic devices that provide voice, video or data communication. Further, while the computer system 400 is illustrated in the singular, the term “system” shall also be taken to include any collection of systems or sub-systems that individually or jointly execute a set, or multiple sets, of software instructions to perform one or more computer functions.

As illustrated in FIG. 4, the computer system 400 includes a processor 410. The processor 410 may be considered a representative example of the processor 152 of the controller 150 in FIG. 1C and executes instructions to implement some or all aspects of methods and processes described herein. The processor 410 is tangible and non-transitory. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The processor 410 is an article of manufacture and/or a machine component. The processor 410 is configured to execute software instructions to perform functions as described in the various embodiments herein. The processor 410 may be a general-purpose processor or may be part of an application specific integrated circuit (ASIC). The processor 410 may also be a microprocessor, a microcomputer, a processor chip, a controller, a microcontroller, a digital signal processor (DSP), a state machine, or a programmable logic device. The processor 410 may also be a logical circuit, including a programmable gate array (PGA), such as a field programmable gate array (FPGA), or another type of circuit that includes discrete gate and/or transistor logic. The processor 410 may be a central processing unit (CPU), a graphics processing unit (GPU), or both. Additionally, any processor described herein may include multiple processors, parallel processors, or both. Multiple processors may be included in, or coupled to, a single device or multiple devices.

The term “processor” as used herein encompasses an electronic component able to execute a program or machine executable instruction. References to a computing device comprising “a processor” should be interpreted to include more than one processor or processing core, as in a multi-core processor. A processor may also refer to a collection of processors within a single computer system or distributed among multiple computer systems. The term computing device should also be interpreted to include a collection or network of computing devices each including a processor or processors. Programs have software instructions performed by one or multiple processors that may be within the same computing device or which may be distributed across multiple computing devices.

The computer system 400 further includes a main memory 420 and a static memory 430, where memories in the computer system 400 communicate with each other and the processor 410 via a bus 408. Either or both of the main memory 420 and the static memory 430 may be considered representative examples of the memory 151 of the controller 150 in FIG. 1C, and store instructions used to implement some or all aspects of methods and processes described herein. Memories described herein are tangible storage mediums for storing data and executable software instructions and are non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The main memory 420 and the static memory 430 are articles of manufacture and/or machine components. The main memory 420 and the static memory 430 are computer-readable mediums from which data and executable software instructions can be read by a computer (e.g., the processor 410). Each of the main memory 420 and the static memory 430 may be implemented as one or more of random access memory (RAM), read only memory (ROM), flash memory, electrically programmable read only memory (EPROM), electrically erasable programmable read-only memory (EEPROM), registers, a hard disk, a removable disk, tape, compact disk read only memory (CD-ROM), digital versatile disk (DVD), floppy disk, Blu-ray disk, or any other form of storage medium known in the art. The memories may be volatile or non-volatile, secure and/or encrypted, unsecure and/or unencrypted.

“Memory” is an example of a computer-readable storage medium. Computer memory is any memory which is directly accessible to a processor. Examples of computer memory include, but are not limited to RAM memory, registers, and register files. References to “computer memory” or “memory” should be interpreted as possibly being multiple memories. The memory may for instance be multiple memories within the same computer system. The memory may also be multiple memories distributed amongst multiple computer systems or computing devices.

As shown, the computer system 400 further includes a video display unit 450, such as a liquid crystal display (LCD), an organic light emitting diode (OLED), a flat panel display, a solid-state display, or a cathode ray tube (CRT), for example. Additionally, the computer system 400 includes an input device 460, such as a keyboard/virtual keyboard or touch-sensitive input screen or speech input with speech recognition, and a cursor control device 470, such as a mouse or touch-sensitive input screen or pad. The computer system 400 also optionally includes a disk drive unit 480, a signal generation device 490, such as a speaker or remote control, and/or a network interface device 440.

In an embodiment, as depicted in FIG. 4, the disk drive unit 480 includes a computer-readable medium 482 in which one or more sets of software instructions 484 (software) are embedded. The sets of software instructions 484 are read from the computer-readable medium 482 to be executed by the processor 410. Further, the software instructions 484, when executed by the processor 410, perform one or more steps of the methods and processes as described herein. In an embodiment, the software instructions 484 reside all or in part within the main memory 420, the static memory 430 and/or the processor 410 during execution by the computer system 400. Further, the computer-readable medium 482 may include software instructions 484 or receive and execute software instructions 484 responsive to a propagated signal, so that a device connected to a network 401 communicates voice, video or data over the network 401. The software instructions 484 may be transmitted or received over the network 401 via the network interface device 440.

In an embodiment, dedicated hardware implementations, such as application-specific integrated circuits (ASICs), field programmable gate arrays (FPGAs), programmable logic arrays and other hardware components, are constructed to implement one or more of the methods described herein. One or more embodiments described herein may implement functions using two or more specific interconnected hardware modules or devices with related control and data signals that can be communicated between and through the modules. Accordingly, the present disclosure encompasses software, firmware, and hardware implementations. Nothing in the present application should be interpreted as being implemented or implementable solely with software and not hardware such as a tangible non-transitory processor and/or memory.

In accordance with various embodiments of the present disclosure, the methods described herein may be implemented using a hardware computer system that executes software programs. Further, in an exemplary, non-limited embodiment, implementations can include distributed processing, component/object distributed processing, and parallel processing. Virtual computer system processing may implement one or more of the methods or functionalities as described herein, and a processor described herein may be used to support a virtual processing environment.

Accordingly, contactless wound monitoring enables a monitoring device with rapid surface monitoring of infection status by focusing on describing the wound infection status either in a binary form or in a rough quantification. The contactless wound monitoring described herein allows for also monitoring trends in severity of infection over time, which in turn provides valuable information for assessing the success chances of a current treatment strategy and for adjusting the current treatment strategy (e.g. by increasing a drug dose), when appropriate. Contactless wound monitoring may be used to rapidly obtain status of a wound, such as when elaborate information of the nature of any wound infection is not particularly sought. Contactless wound monitoring should minimally impact workflow while serving as a gating process to indicate trends and any need for further investigations. Contactless wound monitoring may be used for home, bedside monitoring, during interventions in the catherization lab, and in a variety of other contexts. In clinical use, contactless wound monitoring may be employed in a variety of fields including peripheral vessel disease, surgical wounds and/or dermatology applications. A single device used for the contactless wound monitoring may be both mobile and portable or may be integrated into a catheterization lab such as by integrating the device with a workstation.

Although contactless wound monitoring has been described with reference to several exemplary embodiments, it is understood that the words that have been used are words of description and illustration, rather than words of limitation. Changes may be made within the purview of the appended claims, as presently stated and as amended, without departing from the scope and spirit of contactless wound monitoring in its aspects. Although contactless wound monitoring has been described with reference to particular means, materials and embodiments, contactless wound monitoring is not intended to be limited to the particulars disclosed; rather contactless wound monitoring extends to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72 (b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. A device, comprising:
a light source that emits emission light to a wound at a plurality of wavelengths;
a camera that detects backscattered light generated based on the emission light;
a memory that stores instructions; and
a processor that executes the instructions, wherein, when executed by the processor, the instructions cause the device to:
record an image based on the backscattered light;
quantify a degree of infection of the wound based on the image, and
output an indication of the degree of infection.

2. The device of claim 1, further comprising:
an interface used to output at least one of a visual signal or an audible signal as the indication of whether the wound is infected.

3. The device of claim 1, further comprising:
a filter that filters the backscattered light before the device records the image.

4. The device of claim 1, further comprising:
an interface used to select a range of wavelengths of the backscattered light to use in detecting the backscattered light.

5. The device of claim 1, wherein, when executed by the processor, the instructions cause the device further to:
apply a regression model to the image; and
quantify the degree of infection based on an output of the regression model.

6. The device of claim 1, further comprising:
a thermographic sensor configured to capture a thermographic profile of the wound.

7. A method for quantifying a degree of an infection, comprising:
emitting, by a light source of a device, emission light to a wound at a plurality of wavelengths;
detecting, by a camera of the device, backscattered light generated based on the emission light;
recording an image based on the backscattered light;
quantifying a degree of infection of the wound based on the image, and
outputting, from the device, an indication of whether the wound is infected.

8. The method of claim 7, further comprising:
at least one of displaying a visual signal or outputting an audible signal as the indication of whether the wound is infected.

9. The method of claim 7, further comprising:
filtering the backscattered light before recording the image.

10. The method of claim 7, further comprising:
accepting a selection of wavelengths of the backscattered light to use in recording the image.

11. The method of claim 7, further comprising:
applying a regression model to the image; and
quantifying the degree of infection of the wound based on an output of the regression model.

12. The method of claim 7, wherein outputting the indication of whether the wound is infected comprises outputting an indication of whether the wound is infected for each of a plurality of subsets of pixel values used to generate the image.

13. The method of claim 7, further comprising:
emitting the emission light to a surface other than the wound, detecting backscattered light generated based on the emission light emitted to the surface other than the wound, and recording a reference image based on the surface other than the wound.

14. The method of claim 7, wherein the output of the indication of whether the wound is infected is output as a time-series of a plurality of indications of whether the wound is infected over a period of time.

15. The method of claim 7, further comprising:
normalizing a spectrum of the backscattered light before recording the image based on the backscattered light.
